SEQUENCE LISTING (1 sequence)

SEQ ID NO: 1 - protein, 22 aa, synthetic construct
GFYPSDIAVEWESNGQPENNYK

(19) United States
(12) Patent Application Publication
(10) Pub. No.: US 2026/0229474 A1
(43) Pub. Date: Aug. 6, 2026

(54) DEAMIDATION DETERMINATION USING DIRECT INFUSION MASS SPECTROMETRY

(71) Applicant: Wisconsin Alumni Research Foundation, Madison, WI (US)

(72) Inventors: Joshua Coon, Middleon, WI (US); Austin Salome, Madison, WI (US); Craig Wenger, Sunnyvale, CA (US); Keaton Mertz, La Jolla, CA (US)

(21) Appl. No.: 19/530,148

(22) Filed: Feb. 4, 2026

Related U.S. Application Data
(60) Provisional application No. 63/754,418, filed on Feb. 5, 2025.

(51) Int. Cl. H01J 49/00; G01N 33/68; H01J 49/04

(52) U.S. Cl. CPC H01J 49/0036; G01N 33/6848; H01J 49/0431; G01N 2440/16

(57) ABSTRACT

The present invention provides methods and compositions for detecting and characterizing the amount of deamidation within polypeptides using direct infusion mass spectrometry. The methods of the present invention are able to be performed without having to perform additional liquid or gas chromatography, salt removal steps, or other similar separation techniques on the polypeptides prior to injection into the mass spectrometer device. The methods and systems described herein have the potential to significantly increase peptide analysis and detection of deamidation while providing accuracy and sequence coverage comparable to conventional protein mapping methods.

### CROSS-REFERENCE TO RELATED APPLICATIONS

This application claims priority from U.S. Provisional Patent Application No. 63/754,418, filed Feb. 5, 2025, which is incorporated by reference herein to the extent that there is no inconsistency with the present disclosure.

### STATEMENT REGARDING FEDERALLY SPONSORED RESEARCH

This invention was made with government support under GM118110 and GM108538 awarded by the National Institutes of Health. The government has certain rights in the invention.

### REFERENCE TO AN ELECTRONIC SEQUENCE LISTING

The contents of the electronic sequence listing (94-24.xml; Size: 1,748 bytes; and Date of Creation: Feb. 4, 2026) is herein incorporated by reference in its entirety.

### BACKGROUND OF THE INVENTION

Biotherapeutic proteins are vital medicines for a wide range of diseases with extracellular targets (Dumontet et al., Nature Reviews Drug Discovery 2023, 22 (8):641-661; Li et al., Nature Reviews Drug Discovery 2023, 22 (6): 449-475; Oostindie et al., Nature Reviews Drug Discovery 2022, 21 (10): 715-735; and Swain et al., Nature Reviews Drug Discovery 2023, 22 (2): 101-126). These protein-based drugs, however, sometimes acquire deleterious modifications during production and storage that can impact safety and efficacy (Wei et al., Drug Discovery Today 2014, 19 (1): 95-102; Yang et al., Mabs 2023, 15 (1): p. 2197668; and Federici et al. Biologicals 2013, 41 (3): 131-147).

For each drug, the production, formulation, and storage conditions must be optimized for stability to ensure molecular fidelity. Numerous assays are implemented to monitor their quality. Peptide mapping generates site specific modification levels used to identify critical quality attributes (CQAs). Peptide mapping is especially powerful for the analysis of monoclonal antibodies (mAbs) because their sequences typically only differ from one another in specific regions (Federici et al., Biologicals 2013, 41 (3): 131-147; and Lund et al., Journal of Pharmaceutical Sciences 2021, 110 (2): 619-626). Understanding the specific residues that are prone to modifications that affect efficacy is essential for all stages of mAbs development from discovery, through development and formulation, to production.

Conventional peptide mapping typically entails liquid chromatography (LC) coupled with mass spectrometry (MS) analysis of enzymatically produced peptides from the target protein. Peptides composing the complete sequence and common chemical and biological modifications (e.g., oxidation, deamidation, isomerization, glycosylation, clipping, etc.) are observed. The signals for the modified and native (unmodified) peptides are compared to generate and estimate of the modification stoichiometry. These measurements can be correlated with bioactivity/binding analyses to inform strategies for production, formulation, and storage of biotherapeutic proteins to specifically protect the critical amino acids.

Demand for peptide mapping analysis is growing substantially due to both the power of the technique as well as an increase in the number of biotherapeutic proteins being developed commercially. However, the collection of peptide mapping data is both cumbersome and complicated, requiring long data collection times. The throughput of peptide mapping is inherently limited by the requirement to use chromatography. Short chromatographic methods can increase throughput, but at the cost of decreased resolution that can prevent the detection of deamidated peptides, which are often difficult to separate from their native (unmodified) forms. Additionally, chromatographic columns must be equilibrated for extended times prior to separation and may require blank runs after every analysis to limit carryover.

Deamidation is a chemical reaction in which an amide functional group, such as in the side chain of an amino acid, is removed or is converted to another functional group. For example, deamidation can convert asparagine into aspartic acid or isoaspartic acid, and can convert glutamine into glutamic acid or pyroglutamic acid (5-oxoproline). Deamidation degrades proteins by damaging the amide-containing side chains of the amino acids, and can also affect the biological function, pharmacokinetic properties, and immunogenicity of therapeutic proteins. Detecting deamidation of polypeptides can be challenging due to the subtle mass change that occurs. Deamidation typically adds a 0.984 Da mass shift to the polypeptide; however, this mass shift is only 19 mDa different from the native peptide containing a ¹³C isotope. Thus, these species are indistinguishable under most mass spectrometry conditions.

Accordingly, what is needed are improved methods that permit faster MS analysis but that also enable the determination of deamidation in proteins, polypeptides, and other molecules.

### SUMMARY OF THE INVENTION

The present invention provides methods and compositions for detecting deamidated amino acid residues using mass spectrometry (MS), and for quantifying the amount of deamidation in polypeptides using MS analysis. Preferably, the methods of the present invention are able to analyze therapeutic proteins and antibodies without having to perform liquid or gas chromatography, salt removal steps, or other similar separation techniques.

In one aspect, the present invention provides a method for preparing and analyzing polypeptides via direct infusion mass spectrometry (MS) to obtain MS data, which is further analyzed to characterize the amount of deamidation within the polypeptides. “Direct infusion”, also known as “direct injection”, refers to the direct analysis of a sample by mass spectrometry with little to no separation or purification steps, such as liquid chromatography, being performed on analytes within the sample prior to the sample being inserted into the MS device. In an embodiment, direct infusion is performed by use of electrospray ionization where a liquid sample is infused into an electrospray emitter directly. The sample is then ionized by electrospray ionization and the ions are then subsequently measured by mass spectrometry.

Preferably, the sample is analyzed for the presence of deamidation without the use of liquid chromatography (LC) or other similar separation techniques prior to MS analysis. In an embodiment, the sample is a mixture of polypeptides resulting from a proteolytic digest of a single protein or simple protein mixture, including but not limited to a therapeutic protein, a monoclonal antibody (mAb) or antibody drug conjugate. The mixture contains one or more polypeptides having an unmodified amide functional group, a deamidated amide functional group, or combinations of unmodified and deamidated amide functional groups. Following direct infusion, m/z peaks in the resulting mass spectrum are detected for the polypeptides of interest, and the resulting MS data is analyzed to distinguish polypeptides having deamidated amide functional groups from polypeptides having unmodified amide functional groups, including unmodified polypeptides containing heavy or light isotopes. The removal of conventional chromatography and other separation steps can significantly improve the time and costs requirements of such analyses.

One embodiment of the invention provides a method for analyzing a polypeptide having one or more amino acid residues comprising an unmodified amide functional group or a deamidated amide functional group. The method comprising the steps of: a) injecting the polypeptide into an ion source of a mass spectrometer device; b) generating one or more precursor ions from the polypeptide injected into the ion source of the mass spectrometer device; c) performing one or more MS scans on the one or more precursor ions generated from the polypeptide and obtaining mass spectrometry data on the polypeptide; and d) characterizing the presence and/or amount of deamidated amide functional groups in the polypeptide based on the mass spectrometry data. The mass spectrometry data includes measuring a mass-to-charge ratio and/or signal intensity of the one or more precursor ions and/or one or more fragment ions thereof.

In an embodiment, characterizing the presence and/or amount of deamidated amide functional groups in the polypeptide comprises the steps of: i) measuring a signal intensity of the one or more precursor ions; ii) calculating a theoretical isotopic abundance of the one or more amino acid residues containing the unmodified amide functional group in the polypeptide, and calculating a theoretical isotopic abundance of the one or more amino acid residues comprising the deamidated amide functional group in the polypeptide; iii) determining a contribution of the one or more amino acid residues comprising the unmodified amide functional group in the polypeptide to the signal intensity of the one or more precursor ions; and iv) determining a contribution of the one or more amino acid residues comprising the deamidated amide functional group in the polypeptide to the signal intensity of the one or more precursor ions.

Optionally, the theoretical isotopic abundance for the one or more amino acid residues comprising the deamidated amide functional group are not calculated. The ratio of the unmodified (i.e., native) amino acid residues to the deamidated amino acid residues are determined by solving for the ratio of the unmodified polypeptide or amino acid residues and the one or more shifted versions of the polypeptide or amino acid residues (shifted by 0.984 Da/z).

In an embodiment, characterizing the presence and/or amount of deamidated amide functional groups in the polypeptide comprises the steps of: i) measuring a signal intensity of the one or more precursor ions; ii) calculating a theoretical isotopic abundance of the one or more amino acid residues containing the unmodified amide functional group in the polypeptide; and iii) determining a contribution of the one or more amino acid residues comprising the unmodified amide functional group in the polypeptide to the signal intensity of the one or more precursor ions.

In an embodiment, characterizing the presence and/or amount of deamidated amide functional groups in the polypeptide comprises the steps of: i) measuring a signal intensity of the one or more precursor ions; ii) calculating a theoretical isotopic abundance of the one or more amino acid residues comprising the deamidated amide functional group in the polypeptide; and iii) determining a contribution of the one or more amino acid residues comprising the deamidated amide functional group in the polypeptide to the signal intensity of the one or more precursor ions.

In a further embodiment, the contribution to the signal intensity by the one or more precursor ions having the unmodified and/or deamidated amide functional groups is determined by performing a decomposition method (also sometimes referred to as a deconvolution method) on the obtained mass spectrometry data. Preferably, the obtained mass spectrometry data comprises the measured signal intensity of the one or more precursor ions and the calculated theoretical isotopic abundances of the one or more amino acid residues. In an embodiment, the performed decomposition method comprises a non-negative least squares method, a non-negative least absolute deviation regression method, a general optimizer with additional constraints method, an iterative least squares approach method whenever part of the solution is negative, or a convex optimization method having a non-negativity constraint.

In an embodiment, at least a portion of the precursor ions are fragmented, thereby generating product ions (also referred to as fragment ions). The mass-to-charge ratios and/or signal intensity of the product ions are measured to generate product ion mass spectrometry data. The relative abundance of product ions containing unmodified amide functional groups, deamidated amide functional groups, and combinations thereof, are determined from the product ion mass spectrometry data. In a further embodiment, product ions containing deamidated residues are identified, which also allows the amino acid residues containing deamidated functional groups in the initial polypeptide to be identified.

An additional embodiment of the invention provides methods for preparing a polypeptide or a plurality of polypeptides containing unmodified or deamidated amide functional groups using a method that enables rapid MS analysis compatible with direct infusion and without requiring additional purification or separation steps. This method is also beneficial in that it does not remove small polypeptides that are often removed during chromatography or other separation steps, thereby allowing MS detection and analysis of these polypeptides.

In an embodiment, the method comprises denaturing the polypeptide, treating the denatured polypeptide with a reducing agent, treating the reduced polypeptide with a first solution causing the reduced polypeptide to precipitate out of the first solution, resuspending the polypeptide precipitate in a second solution, and injecting the resuspended polypeptide into the ion source of the mass spectrometer device. As a result, the resuspended polypeptide may be injected into the mass spectrometry device without performing a liquid chromatography step, an additional salt removal step, and/or any other additional separation steps.

The denaturing step comprises treating the polypeptide with a chemical agent able to denature proteins and polypeptides as is known in the art. In an embodiment, the denaturing step comprises contacting each of the polypeptide with one or more of guanidinium chloride, urea, a detergent, or solutions thereof. Preferably, the polypeptide is contacted with a solution containing of guanidinium chloride or urea.

The reducing agent may be any chemical agent known in the art able to reduce the denatured polypeptides, including but not limited to dithiothreitol (DTT) and solutions thereof. Optionally, the one or more denatured polypeptides or reduced polypeptides are additionally treated with an alkylating agent. The alkylating agent may be any alkylating agent known in the art suitable for use with polypeptides including, but not limited to, iodoacetate, iodoacetic acid, iodoacetamide (all referenced herein as IAA) and solutions thereof.

As used herein, the first solution may be any chemical agent able to cause polypeptides, particularly the reduced polypeptides, to precipitate out of solution, including acids, organic solvents, salts, and metals as is known in the art. Preferably, the first solution comprises an organic solvent, such as methanol (MeOH).

As used herein, a wash solution is optionally used to wash the one or more polypeptide precipitates in order to reduce or remove salts and other unwanted contaminants. In an embodiment, the wash solution comprises water, MeOH, and combinations thereof. Preferably, the second solution comprises 70%-95% MeOH, such as a 20/80 or 10/90 water to MeOH mixture.

In an embodiment, a second solution is used to dissolve the one or more polypeptide precipitates back into solution, and may be any solution able to dissolve the polypeptide precipitates, including but not limited to ammonium acetate, ammonium bicarbonate, and other buffer solutions.

The methods described herein are advantageous in that they can prepare polypeptides for MS analysis in a sufficiently purified form so as to allow polypeptides to be injected into a MS device without requiring additional salt removal steps and/or chromatography steps. For example, in an embodiment, the method does not comprise an additional salt removal step other than what is achieved by washing the polypeptide precipitates with the wash solution. In a similar embodiment, the method does not comprise a liquid chromatography step and/or or other additional separation step.

Optionally, the polypeptide is generated by digesting a polypeptide precursor (i.e., a larger polypeptide or a protein). Digestion of the polypeptide precursor may be performed using one or more digestion enzymes or chemical agents as is known in the art. In an embodiment, the digestion step comprises contacting each of the dissolved polypeptides with a proteolytic enzyme, including but not limited to trypsin, pepsin, chymotrypsin, papain, calpain, serrapeptase, thermolysin, carboxypeptidase, and combinations thereof. Optionally, an acid quenching step is performed on the resulting digested polypeptides by adding an acidic solution, including but not limited to a formic acid solution, to the digested polypeptides.

In an embodiment, the polypeptide is a biotherapeutic (or portions thereof), including but not limited to antibodies and/or therapeutic proteins. Preferably, a specific sequence or region of a control polypeptide or polypeptide standard is known, such as a region of an antibody that affects activity or stability of the antibody, or a region that is susceptible to degradation or modification. Accordingly, a further embodiment of the invention comprises comparing one or more specific regions of each of the plurality of polypeptides with a corresponding region of a control polypeptide and determining if a chemical modification is present in one or more of the plurality of polypeptides.

Preferably, multiple MS scans of each precursor ion are performed. The MS scans may be a full mass range scan (which provides a full mass spectrum of each analyte), a narrow mass range scan (which provides a mass spectrum over a specified m/z range), a segmented mass range scan, or combinations thereof. As used herein, “maximum spectral dynamic range” is the ratio of the largest and smallest signal that can be detected in a mass spectrum. With a segmented mass range scan, the mass range of interest is broken into two or more segments, where each segment is scanned in succession and the resulting scans are combined together. This increases the combined-spectra effective-dynamic-range, which means the ratio of the largest signal divided by the smallest signal in a mass spectrum can be increased. This allows a wider range of ion intensities to be detected meaning smaller signal can still be detected in the presence of large signals. In an embodiment, the sample is a liquid sample that is directly injected into an electrospray emitter of an MS device, including but not limited to an Orbitrap MS device. Preferably, the MS device provides a resolving power equal to or greater than 100,000, has a scan rate of at least 0.5 scans per second (0.5 Hz), and the multiple scans are performed over a time period of at least 10 seconds. Additionally, the method preferably does not comprise any additional liquid or gas chromatography steps or any additional salt removal steps.

The mass spectrometry data may include mass-to-charge ratios and/or signal intensity obtained from MS1 data, MS2, or both. Optionally, the spectral data is generated by averaging data obtained from multiple scans for each precursor ion. Site specific deamidation, optionally with site specific mutations, post translational modifications, or combinations thereof, are detected and/or measured in the polypeptides. Optionally, the biological activity of each of the polypeptides is measured and a link between the biological activity and the site specific deamidation are recorded for each polypeptide. Comparing the generated precursor ion or product ion mass spectrometry data to the predicted ion data of the polypeptide can confirm the spectral data and can confirm the sequence order of the polypeptides that generate the precursor ions, which can provide sequence confirmation.

The method of the present invention also enables relative quantification of the polypeptides in the sample. The peak intensity of two or more detected precursor ions in MS1 events or their product ions in MS/MS events are compared to each other and the relative abundance in the sample of each polypeptide generating the precursor ions is then calculated from the compared peak intensities. Preferably, the polypeptides generating the two or more detected precursor ions comprise a first polypeptide and a variant of the first polypeptide having a modified amino acid sequence, a modified structure, or a modified chemical formula. As a result, the relative abundance of the first polypeptide to the modified polypeptide in the sample is able to be calculated. Such modifications to the polypeptide include, but are not limited to, oxidation, deamidation, isomerization, glycosylation, substitutions of amino acids, and clipping. For example, specific regions a manufactured antibody or therapeutic protein may be compared to a control protein or antibody to determine if the manufactured antibody or therapeutic protein has undergone a modification that would affect the stability or activity of the molecule. In an embodiment, one of the polypeptides is a known peptide standard.

Optionally, the MS device provides a resolving power equal to or greater than 100,000, or equal to or greater than 200,000, or equal to or greater than 480,000, or equal to or greater than 960,000. Additionally, the MS device preferably has a scan rate of at least one scan per second (1 Hz), at least two scans per second (2 Hz), at least five scans per second (5 Hz), or at least ten scans per second (10 Hz). In many conventional MS methods using liquid chromatography, the MS device has a time period of approximately 6-10 seconds to scan the analytes eluting from the chromatography column before the analytes are moved to make room for the next batch of analytes. However, using the direct infusion method of the present invention allows the MS device to perform multiple scans over a time period of at least 5 seconds, over a time period of at least 10 seconds, over a time period of at least 20 seconds, over a time period of at least 30 seconds, over a time period of at least 45 seconds, over a time period of at least 60 seconds, over a time period of at least 90 seconds, or over a time period of at least 120 seconds.

In an embodiment, at least 50 samples, each generating a plurality of polypeptides, are analyzed within 24 hours using the above methods on a single mass spectrometry device, preferably at least 100 samples within 24 hours, preferably at least 150 samples within 24 hours, preferably at least 200 samples within 24 hours, preferably at least 250 samples within 24 hours, preferably at least 300 samples within 24 hours, preferably at least 400 samples within 24 hours, preferably at least 500 samples within 24 hours, or more preferably at least 600 samples within 24 hours. In an embodiment, each sample is analyzed at an average time of ≤5.0 min per sample, preferably ≤4.5 min per sample, preferably ≤4.0 min per sample, preferably or ≤3.0 min per sample. Optionally, each sample is an antibody or therapeutic protein that is digested to generate a plurality of polypeptides.

### BRIEF DESCRIPTION OF THE DRAWINGS

FIG. 1 shows an overview of current peptide mapping and technology.

FIG. 2 illustrates technology (see PCT/US24/17865 to Coon et al.) to accelerate peptide mapping by 100-fold using direct infusion that does not require liquid chromatography.

FIG. 3 LC-MS trace of the GFYPSDIAVEWESNGQPENNYK (SEQ ID NO:1) peptide showing the LC trace as well as the mass spectrum of the native and deamidated species.

### DETAILED DESCRIPTION OF THE INVENTION

### Definitions

In general the terms and phrases used herein have their art-recognized meaning, which can be found by reference to standard texts, journal references and contexts known to those skilled in the art. The following definitions are provided to clarify their specific use in the context of the invention.

An “amide functional group” refers to an organic functional group having a nitrogen atom attached to a carbonyl carbon atom. As used herein, amide function groups include simple amides having the general formula:

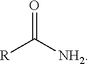

Deamidation is a chemical reaction in which an amide functional group, such as in the side chain of an amino acid, is removed or is converted to another functional group, such as to a carboxylic acid.

The terms “native peptide” and “unmodified peptide” refer to a peptide or polypeptide having an amide functional group that has not undergone deamidation.

As used herein, the term “precursor ion” is used herein to refer to an ion which is produced during ionization stage of mass spectrometry analysis, including the MS1 ionization stage of MS/MS analysis.

As used herein, the term “product ion” is used to refer to an ion which is produced during a fragmentation process of a precursor ion, including the fragmentation stage of MS/MS analysis.

As used herein, the term “mass spectrometry” (MS) refers to an analytical technique for the determination of the elemental composition of an analyte, including but not limited to polypeptides. The mass spectrometry principle consists of ionizing analytes to generate charged species (i.e., precursor ions) or species fragments (i.e., product ions) and measurement of their mass-to-charge ratios. Conducting a mass spectrometric analysis of an analyte results in the generation of mass spectrometry data relating to the mass-to-charge ratios of the analyte and analyte product ions. Mass spectrometry data corresponding to analyte ion and analyte ion fragments is presented in mass-to-charge (m/z) units representing the mass-to-charge ratios of the analyte precursor ions and/or analyte product ions. In tandem mass spectrometry (MS/MS, MS2, or MSn), multiple rounds of mass spectrometry analysis are performed. For example, samples containing a mixture of polypeptides can be ionized and the resulting precursor ions separated according to their mass-to-charge ratio. Selected precursor ions can then be fragmented and further analyzed according to the mass-to-charge ratio of the product ions.

As used herein, the term “mass spectrometer” refers to a device which creates ions from a sample, separates the ions according to mass to charge ratios, and measures the signal intensity of each detected m/z peak. Because charge (z) can be calculated from the spectra, mass can be determined. Mass spectrometers include multistage mass spectrometers which fragment the mass-separated precursor ions and separate the product ions by mass-to-charge ratio one or more times. Multistage mass spectrometers include tandem mass spectrometers which fragment the mass-separated precursor ions and separate the product ions by mass-to-charge ratio once.

As used herein, the term “ion source” refers to a component of a mass spectrometer which produces ions from a sample. Examples of ion sources include, but are not limited to, electrospray ionization (ESI) sources, matrix assisted laser desorption/ionization (MALDI) sources, atmospheric pressure chemical Ionization (APCI) sources, and atmospheric pressure photoionization (APPI) sources.

As used herein, the term “mass-to-charge ratio” refers to the ratio of the mass of a species (i.e., a precursor ion or product ion) or to the charge state of a species. The term “m/z unit” refers to a measure of the mass to charge ratio. The Thomson unit (abbreviated as Th) is an example of an m/z unit and is defined as the absolute value of the ratio of the mass of an ion (in Daltons) to the charge of the ion (with respect to the elemental charge).

“Mass spectrometer resolving power”, often termed resolution, is a quantitative measure of how well m/z peaks in a mass spectrum are separated (i.e., resolved). There are a variety of conventions to calculate resolving power. The IUPAC definition is: Resolving power (R): R=m/Δm. This equation illustrates how to calculate resolving power (R) where m is the mass corresponding to the peak and Δm is the spacing between that peak and the nearest neighbor peak. Another, definition for resolving power is: Resolving power (R): R=m/(m½), where m is the mass corresponding to the peak (m) and m½ is a variable referring to the full width at half maximum of the peak (m½=FWHM). With the second definition, two peaks at m/z 500 and 501 are just barely discernible if the resolving power is 500. This method of calculating resolution is particularly useful as it provides a metric to assess peak width regardless of whether there is a nearby neighbor to compare it to.

The term “protein” refers to a class of compounds comprising one or more polypeptide chains and/or modified polypeptide chains. Proteins can be modified by naturally occurring processes such as post-translational modifications or co-translational modifications. Exemplary post-translational modifications or co-translational modifications include, but are not limited to, phosphorylation, glycosylation, deamidation, lipidation, prenylation, sulfonation, hydroxylation, acetylation, methylation, methionine oxidation, the addition of cofactors, proteolysis, and assembly of proteins into macromolecular complexes. Modification of proteins can also include non-naturally occurring derivatives, analogues and functional mimetics generated by chemical synthesis. Exemplary derivatives include chemical modifications such as alkylation, acylation, carbamylation, iodination or any modification that derivatizes the protein.

The terms “peptide” and “polypeptide” are used synonymously in the present description, and refer to a class of compounds composed of amino acid residues chemically bonded together by amide bonds (or peptide bonds). Peptides and polypeptides are polymeric compounds comprising at least two amino acid residues or modified amino acid residues. Modifications can be naturally occurring or non-naturally occurring, such as modifications generated by chemical synthesis. Modifications to amino acids in peptides include, but are not limited to, phosphorylation, glycosylation, deamidation, lipidation, prenylation, sulfonation, hydroxylation, acetylation, methylation, methionine oxidation, alkylation, acylation, carbamylation, iodination and the addition of cofactors. Peptides and polypeptides can be generated by substantially complete digestion or by partial digestion of proteins.

### Overview

The present invention provides an alternative to LC-MS based protein mapping assays used to detect deamidation in therapeutic proteins, antibodies, and other polypeptides. The methods and systems described herein have the potential to significantly increase peptide analysis and detection of deamidation while providing accuracy and sequence coverage comparable to conventional protein mapping methods.

Peptide mapping is a ubiquitous method used to monitor critical quality attributes of proteins and polypeptides. Of the many attributes and modifications that are monitored, deamidation monitoring is important as this modification alters both the charge state and structure of the peptide. Deamidation is a common modification that occurs in proteins, such as where the amino acid asparagine is converted to aspartic acid, or where glutamine is converted to glutamic acid. Deamidation typically adds a mass shift to the peptide that is difficult to distinguish from other forms of the peptide that have not undergone deamidation, such as unmodified peptides that contain naturally occurring isotopes.

For example, deamidation converting asparagine (N) to aspartic acid (D) typically adds a 0.984 Da mass shift to the peptide, however, this mass shift is only 19 mDa different from the first ¹³C isotope of the native peptide. These species will be indistinguishable under most mass spectrometry conditions. Traditionally, long LC-MS gradients are used to resolve these species; however, such traditional LC-MS methods can require very long with run times that causes a large bottleneck when running samples.

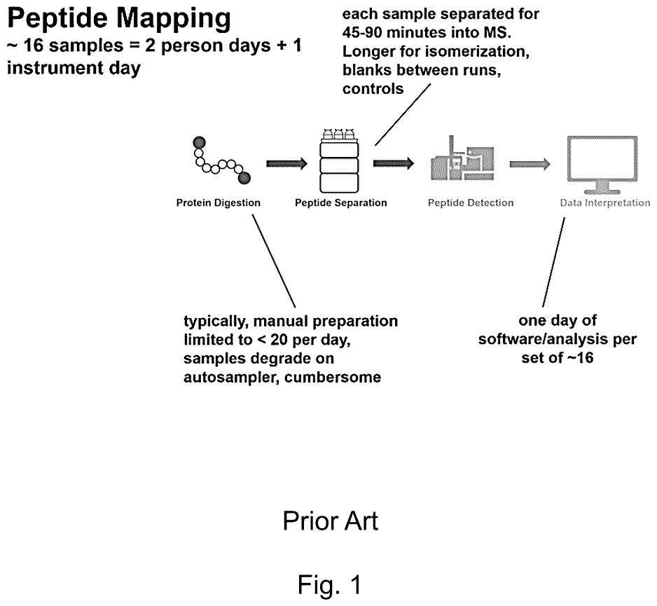

For example, in conventional peptide mapping (illustrated in FIG. 1), proteins are digested into multiple constituent polypeptides by chemical or enzymatic reactions and are then separated, such as by liquid chromatography (LC). A reliable separation platform (i.e., an LC column) should be used that is able to deliver high resolution between hundreds of polypeptides resulting from the digestion. Conventional peptide separation requires about 45-90 minutes per sample, although longer times are required to separate polypeptides having isomerized amino acids residues (such as asparagine and aspartic acid). Following peptide separation, the polypeptides are analyzed using mass spectrometry (MS) to detect the polypeptides of interest. Using appropriate software, conventional peptide mapping systems are typically able to analyse between 15-20 samples per day (approximately 4,000 samples a year per machine) at a cost of over $400 per sample.

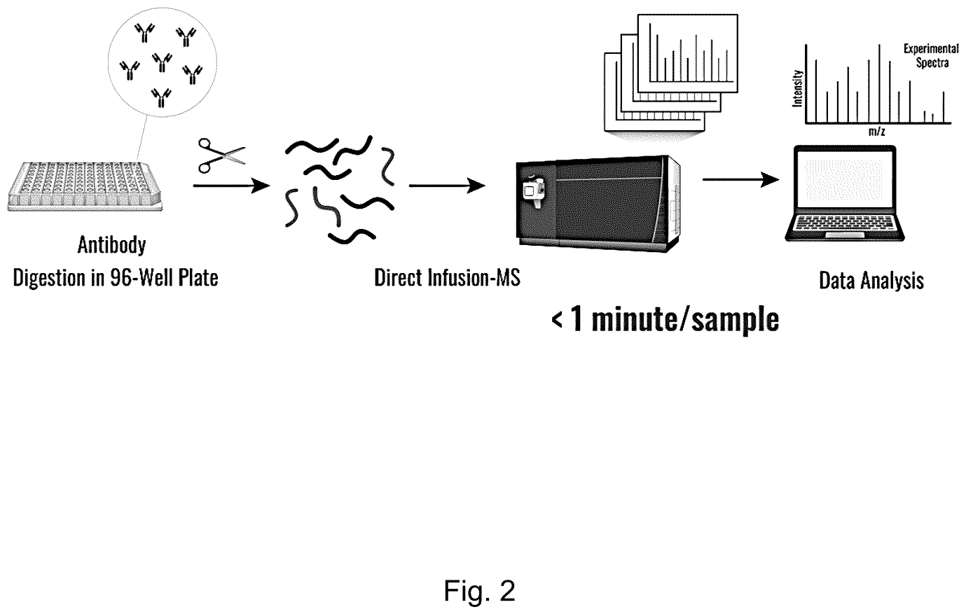

In order to improve speed and efficiency, additional methods (see WO 2024/182604 to Coon et al.) have been developed for peptide mapping that directly analyzes digested antibodies and other therapeutic proteins through direct infusion into the mass spectrometry device without having to perform liquid chromatography. One such method (illustrated in FIG. 2) leverages a plate-based sample preparation compatible with direct infusion MS analysis and is capable of rapidly preparing and analyzing 96 samples within 8 hours. With direct infusion, MS information can be achieved in a couple minutes of instrument time compared to multiple hours which allows for high throughput peptide mapping. However, since LC separation is not used, additional methods are required to determine and characterize the presence of deamidation in the peptides.

Described below are MS data analysis techniques able to determine the amount of deamidation in polypeptide samples analyzed using direct infusion MS. These techniques allow for deamidation information to be obtained by MS1 or MS2 with direct infusion, significantly decreasing data collection time. These techniques are able to work directly with current direct infusion MS methods and devices and provide different ways of determining deamidation based on the instrument and type of MS analysis used.

### EXAMPLES

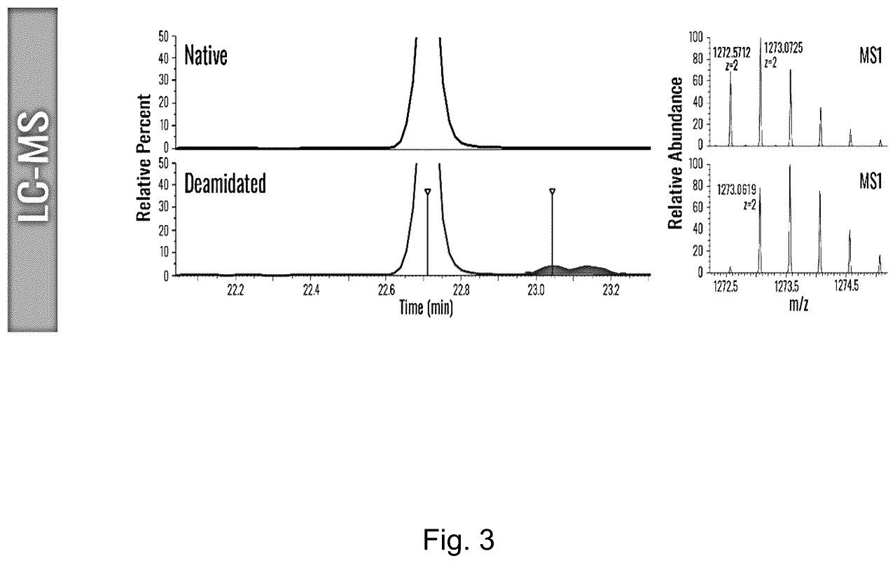

The detection and determination of deamidation in peptides can be challenging due to the subtle mass change that occurs. As previously stated, deamidation often adds a 0.984 Da mass shift to the peptide. However, this mass shift is only 19 mDa different from the native peptide containing a ¹³C isotope. As a result, these species will be indistinguishable under most mass spectrometry conditions. FIG. 3 shows this subtle change in mass with the native peptide on the top along with its mass spectrum followed by the LC trace and spectrum of the deamidated species.

However, if sufficient MS data is collected on the peptide mixture, it is possible to determine the presence of the deamidated peptide and quantify the amount of deamidation. For example, consider the doubly charged form of the peptide GFYPSDIAVEWESNGQPENNYK (SEQ ID NO:1). The theoretical isotopic distribution of the native form can be calculated in silico as illustrated in Table 1 below:

TABLE 1

| | | Relative |
|---|---|---|
| Isotope | | Abundance |
| Number, i | m/z | (%), T_{n, i} |
| 0 | 1272.56933 | 70.55 |
| 1 | 1273.0708 | 100 |
| 2 | 1273.57222 | 75.97 |
| 3 | 1274.07359 | 40.72 |
| 4 | 1274.57493 | 17.17 |
| 5 | 1275.07625 | 6.04 |
| 6 | 1275.57756 | 1.84 |
| 7 | 1276.07885 | 0.49 |
| 8 | 1276.58014 | 0.12 |
| 9 | 1277.08141 | 0.03 |
| 10 | 1277.58272 | 0.01 |

Now consider an unknown fraction of that peptide being deamidated. The theoretical isotopic distribution of the singly deamidated form, regardless of where in the peptide it occurs, can be also calculated in silico:

TABLE 2

| | | Relative |
|---|---|---|
| Isotope | | Abundance |
| Number, i | m/z | (%), T_{d, i} |
| 0 | 1273.06133 | 70.72 |
| 1 | 1273.56282 | 100 |
| 2 | 1274.06424 | 75.96 |
| 3 | 1274.56561 | 40.76 |
| 4 | 1275.06696 | 17.23 |
| 5 | 1275.56828 | 6.08 |
| 6 | 1276.06959 | 1.86 |
| 7 | 1276.57088 | 0.5 |
| 8 | 1277.07217 | 0.12 |
| 9 | 1277.57344 | 0.03 |
| 10 | 1278.07475 | 0.01 |

Note that the mass-to-charge ratio (m/z) of the deamidated form's isotope number 0 is very close to the native form's isotope number 1, deamidated isotope number 1 is very close to native isotope number 2, etc. Because the deamidation mass shift of +0.9840 Da is very similar to the 1.0035 Da mass difference between carbon-12 and carbon-13 isotopes, which dominates peptide isotopic distributions, there is a high degree of overlap. Therefore, fully resolving the native and deamidated isotopic peaks is difficult because even state of the art mass spectrometers do not have sufficient mass resolution. Two separate methods, one MS1-based and one MS2-based, are provided below for quantifying the amount of deamidation.

In an embodiment, it is not necessary to calculate the isotopic distribution for the deamidated version of polypeptide since the isotopic distribution is expected to be similar to the unmodified (i.e., native) form. Solving for the ratio of the unmodified form and versions of the polypeptide which are shifted by 0.984 Da/z can determine the ratio of the unmodified form to the deamidated form. Determining the ratio of the unmodified form to the deamidated form in this manner is simpler than solving the above equations when the Td,i is different for each species.

### Technique 1—MS1 Based Analysis

For the MS¹-based method, intact mass spectra of native and deamidated peptides are acquired. Since the mass resolution is typically not sufficient to resolve deamidated from native isotopic peaks, their abundances are summed together. The observed isotopic peak abundances are a composition of the theoretical isotopic envelopes of both the native and deamidated species.

From here, a series of equations are set up to be solved simultaneously:

$$\{ \begin{matrix}{T}_{n , 0} {x}_{n} = {E}_{0} \\ {T}_{n , 1} {x}_{n} + {T}_{d , 0} {x}_{d} = {E}_{1} \\ {T}_{n , 2} {x}_{n} + {T}_{d , 1} {x}_{d} = {E}_{2} \\ ...\end{matrix}$$

where T_{n,i} is the theoretical isotopic abundance of the ith isotopic peak of the native species, T_{d,i} is the theoretical isotopic abundance of the ith isotopic peak of the deamidated species, xₙ is the non-normalized contribution of the native species, xd is the non-normalized contribution of the deamidated species, and Eᵢ is the experimental abundance of the ith peak. Unknowns xₙ and x_{d} can be solved for. The deamidation occupancy is defined by

$$\frac{{x}_{d}}{{x}_{n} + {x}_{d}} .$$

Since there are more equations than unknowns, the system is overdetermined and a least-squares approach is used to find the optimal solution. Since a negative occupancy is non-sensical, any of the following can be used: non-negative least squares, non-negative least absolute deviation regression, nonlinear least-squares minimization method, a general optimizer with additional constraints, an iterative least squares approach whenever part of the solution is negative, or convex optimization with non-negativity constraint. To improve the solution, each equation is weighted based on abundance, mass accuracy, or other factors which are known to be correlated with accuracy. This method works best with time-of-flight (TOF) mass analyzers that have both sufficient mass resolution to resolve isotopic envelopes and also measure accurate isotopic abundances. However, it can be assumed that the isotopic envelopes are convolved, so this approach can also be used with low resolution mass analyzers such as quadrupole mass filters and ion traps.

A similar mathematical process may be performed for versions of the polypeptide containing multiple deamidation sites. For example, the isotopic distribution for a double deamidated version of the polypeptide may be calculated (or a shifted version of the native may be used as noted above as an approximation) having Tn,i, Td1,i, and Td2,I, and weighting factors xn, xd1, and xd2. The singly deamidated percentage would be xd1/(xn+xd1+xd2), while the total deamidation would be (xd1+xd2)/(xn+xd1+xd2). This process can be continued with more sites of deamidation depending on the quality of the spectrum.

If mass resolution is sufficient, deconvolving the isotopic envelopes is not necessary. One can simply compare the intensity of one or more of the ith isotopes of the native and deamidated species.

If multiple charge states are available, the deamidation occupancy from each is able to be computed separately. A final result is then able to be calculated by averaging or otherwise combining the individual measurements from each charge state.

### Technique 2—MS2 Based Analysis

For the MS2-based method, peptide precursors are isolated and fragmented both with and without the deamidation, and the resulting mass spectrum is recorded with ultrahigh mass resolution. Most peptide fragments are singly charged, which maximizes the separation between native and deamidated isotopic peaks in m/z space, making them more likely to be resolved. Fragment ions, which could potentially contain the deamidation species, are measured to quantify the deamidation at various points along the peptide based on the ratio of abundance of the modified fragment ions to the abundance of both the modified and unmodified fragment ions.

Consider again the peptide GFYPSDIAVEWESNGQPENNYK (SEQ ID NO:1) having a single deamidated functional group. Both the native and deamidated forms are simultaneously co-isolated and co-fragmented. The deamidation occupancy of three asparagine (N) residues are assigned as a, b, and c. Several key fragment ions are labeled:

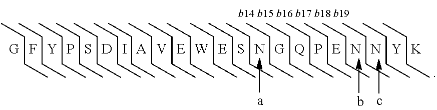

There are five fragment ions (b14-b18) which contain only potential deamidation site, a. Thus, the MS² spectrum can be inspected for the relative abundance of each the resolved deamidated and native fragments to yield an estimated deamidation site occupancy. These site occupancies can be combined to give an overall estimate labeled A. One fragment ion (b19) contains sites a and b; this site occupancy is labeled AB. The same can be done for fragment ions corresponding to C, BC, and ABC. With this information a simultaneous system of equations is used to solve for a, b, and c:

$$\{ \begin{matrix}a = A \\ b = B \\ c = C \\ a + b = AB \\ b + c = BC \\ a + b + c = ABC\end{matrix}$$

Since internal fragments are not typically observed, B cannot be measured. Still, there are 5 equations and only 3 unknowns, making this an overdetermined system, so a similar approach to Technique 1 can be used to find the optimal solution. It must be ensured that the unknowns are not less than zero, so any of the following can be used: non-negative least squares, a general optimizer with additional constraints, or an iterative least squares approach whenever part of the solution is negative. To improve the solution, each equation can be weighted based on abundance, mass accuracy, or other factors which are known to be correlated with accuracy.

This approach works for any peptide with two or more potential deamidation sites, since the number of equations is 2×sites−1. For a peptide with a single potential deamidation site, no system of equations is needed since there is only one equation and one unknown.

Alternatively, as opposed to combining all fragment ions into a single equation, they can be kept as separate equations. In this case, the system of equations would look like this:

$$\{ \begin{matrix}a = {A}_{b 14} \\ a = {A}_{b 15} \\ a = {A}_{b 16} \\ a = {A}_{b 17} \\ a = {A}_{b 18} \\ a + b = {AB}_{b 19} \\ ...\end{matrix}$$

Having now fully described the present invention in some detail by way of illustration and examples for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims. For example, it should be understood that the reagents, solution concentrations, and their amounts used in the above examples may be modified or depending on the specific polypeptides to be analyzed.

When a group of materials, compositions, components or compounds is disclosed herein, it is understood that all individual members of those groups and all subgroups thereof are disclosed separately. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure. Additionally, the end points in a given range are to be included within the range. In the disclosure and the claims, “and/or” means additionally or alternatively. Moreover, any use of a term in the singular also encompasses plural forms.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements.

One of ordinary skill in the art will appreciate that starting materials, device elements, analytical methods, mixtures and combinations of components other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. The invention illustratively described herein suitably may be practiced in the absence of any element or elements, limitation or limitations which is not specifically disclosed herein. Headings are used herein for convenience only.

All publications referred to herein are incorporated herein to the extent not inconsistent herewith. Some references provided herein are incorporated by reference to provide details of additional uses of the invention. All patents and publications mentioned in the specification are indicative of the levels of skill of those skilled in the art to which the invention pertains. References cited herein are incorporated by reference herein in their entirety to indicate the state of the art as of their filing date and it is intended that this information can be employed herein, if needed, to exclude specific embodiments that are in the prior art.

1. A method for analyzing a polypeptide having one or more amino acid residues comprising an unmodified amide functional group or a deamidated amide functional group, the method comprising the steps of:
a) injecting the polypeptide into an ion source of a mass spectrometer device;
b) generating one or more precursor ions from the polypeptide injected into the ion source of the mass spectrometer device;
c) performing one or more MS scans on the one or more precursor ions generated from the polypeptide and obtaining mass spectrometry data on the polypeptide, wherein obtaining mass spectrometry data comprises measuring a mass-to-charge ratio of the one or more precursor ions and/or one or more fragment ions thereof;
d) characterizing the presence and/or amount of deamidated amide functional groups in the polypeptide based on the mass spectrometry data.

2. The method of claim 1, wherein the characterizing step comprises:
i) measuring a signal intensity of the one or more precursor ions;
ii) calculating a theoretical isotopic abundance of the one or more amino acid residues comprising the unmodified amide functional group in the polypeptide; and
iii) determining a contribution of the one or more amino acid residues comprising the unmodified amide functional group in the polypeptide to the signal intensity of the one or more precursor ions.

3. The method of claim 2, wherein determining the contribution to the signal intensity of the one or more precursor ions comprises performing a deconvolution method on the obtained mass spectrometry data,
wherein the obtained mass spectrometry data comprises the measured signal intensity of the one or more precursor ions and the calculated theoretical isotopic abundances of the one or more amino acid residues, and
wherein the performed deconvolution method comprises a non-negative least squares method, a non-negative least absolute deviation regression method, a general optimizer with additional constraints method, an iterative least squares approach method whenever part of the solution is negative, or a convex optimization method having a non-negativity constraint.

4. The method of claim 1, wherein the characterizing step comprises:
i) measuring a signal intensity of the one or more precursor ions;
ii) calculating a theoretical isotopic abundance of the one or more amino acid residues comprising the deamidated amide functional group in the polypeptide; and
iii) determining a contribution of the one or more amino acid residues comprising the deamidated amide functional group in the polypeptide to the signal intensity of the one or more precursor ions.

5. The method of claim 4, wherein determining the contribution to the signal intensity of the one or more precursor ions comprises performing a deconvolution method on the obtained mass spectrometry data,
wherein the obtained mass spectrometry data comprises the measured signal intensity of the one or more precursor ions and the calculated theoretical isotopic abundances of the one or more amino acid residues, and
wherein the performed deconvolution method comprises a non-negative least squares method, a non-negative least absolute deviation regression method, a general optimizer with additional constraints method, an iterative least squares approach method whenever part of the solution is negative, or a convex optimization method having a non-negativity constraint.

6. The method of claim 1 comprising fragmenting at least a portion of the one or more precursor ions, thereby generating product ions, generating product ion mass spectrometry data, and determining the relative abundance of product ions comprising unmodified amide functional groups, deamidated amide functional groups, and combinations thereof.

7. The method of claim 1, wherein injecting the polypeptide into the ion source of the mass spectrometer comprising the steps of: denaturing the polypeptide, treating the denatured polypeptide with a reducing agent, treating the reduced polypeptide with a first solution causing the reduced polypeptide to precipitate out of the first solution, resuspending the polypeptide precipitate in a second solution, and injecting the resuspended polypeptide into the ion source of the mass spectrometer device.

8. The method of claim 1 comprising digesting a polypeptide precursor to generate the polypeptide.

9. The method of claim 1 comprising injecting the polypeptide into the ion source of the mass spectrometer device without performing a liquid chromatography step.

10. The method of claim 1 comprising injecting the polypeptide into the ion source of the mass spectrometer device without performing an additional salt removal step.

11. The method of claim 1, wherein the polypeptide is an antibody.

12. The method of claim 1, wherein the polypeptide is a portion of a polypeptide having or suspected of having one or more asparagine residues modified to be aspartic acid or isoaspartic acid residues.

13. The method of claim 1, wherein the polypeptide is a portion of a polypeptide having or suspected of having one or more glutamine residues modified to be glutamic acid or pyroglutamic acid residues.

14. The method of claim 1, wherein the polypeptide is a therapeutic polypeptide.
